(19) United States
(12) Patent Application Publication
(10) Pub. No.: US 2013/0131698 A1
(43) Pub. Date: May 23, 2013

(54) Antimicrobial Coating for Surgical Implants and Method of Manufacturing

(71) Applicants: John I. Shipp, Atlantic Beach, FL (US); Todd Heniford, Charlotte, NC (US)

(72) Inventors: John I. Shipp, Atlantic Beach, FL (US); Todd Heniford, Charlotte, NC (US)

(21) Appl. No.: 13/299,435

(22) Filed: Nov. 18, 2011

(51) Int. Cl. A61B 17/03; B05D 3/06; B05D 3/00; B05D 7/00; B05D 5/00

(52) U.S. Cl. 606/151; 427/2.24

(57) ABSTRACT

A surgical mesh has a therapeutic amount of an antimicrobial substance attached to the mesh. The surgical mesh is then stabilized by either freeze drying or keeping cold after attaching the antimicrobial substance. The surgical mesh is then sterilized with an electron beam. The stabilization of the surgical mesh allows the mesh to be used up to a year after the antimicrobial substance is attached and still be effective against bacteria.

### BACKGROUND

1. Field of the Invention

The present invention relates generally to an antimicrobial coating for surgical implants, in particular, to antimicrobial coatings for meshes used in hernia and soft tissue repair.

2. Description of the Related Art

It will be appreciated by those skilled in the art that the use of mesh for strengthening hernia and other soft tissue repair such as breast and pelvic floor reconstruction is well known. Synthetic and biological meshes have been implanted for this purpose. Synthetic meshes, generally, cause high inflammatory response and as a result become ineffective and are encapsulated owing to the immune system's foreign body response, often requiring explantation. Decellurized biological materials cause less inflammatory response but are often weaker mechanically. Synthetic materials have no antimicrobial effects to combat infections, a common problem following implant. W. L. Gore produces a product known as DUALMESH® PLUS. The mesh material, expanded PTFE, is coated with two antimicrobial preservative agents, silver carbonate and chlorhexidine diacetate, which act to inhibit bacterial colonization. The FDA classifies these inorganic compounds as toxic materials. Many patients have developed significant fever conditions following implant, consistent with toxic poisoning.

It is believed by many skilled in the art that biological materials provide some antimicrobial effect. As the biological materials degrade, growth factors and peptides are released by the degrading biological materials. The belief is that these released materials possess antimicrobial properties that help ward off potential pathogens. Experience by others has shown that any such antimicrobial properties are inadequate to substantially reduce postoperative infection rates as compared to synthetic antimicrobials.

Approximately ninety percent of post hernia repair infections are caused by Staphylococcus aureus, a gram positive pathogen. Other pathogens, both gram positive and gram negative have, to, a lesser extent, been cultured from hernia infections.

Antimicrobial substances have been impregnated in implant devices such as central venous catheters and other transdermal devices. Bayston, in U.S. patent application 20070224243 discloses such impregnated devices and methods of making them. These devices are inserted through relatively small percutaneous access points, and thus infection sources can continually enter the body from the hospital environment. To be effective, an antimicrobial agent associated with this type implant must last as long as the implant is in place and often must contain multiple agents that are effective against mutations. Because of the size of the opening, the pathogen loads are small (as compared to large surgical openings like open hernia repair wound sites), but continuous. Bayston discloses an impregnation method that slowly leaches out a mixture of antimicrobial agents that is effective up to 180 days. This impregnation method is ineffective for hernia procedures, however, partially because of the difference in the implant material and partially because of the magnitude of the bacterial challenge. Li, in U.S. Pat. No. 6,299,651 discloses the use of an antibacterial effusing textile fabric used to make clothing and other ware such as napkins The process described therein produces an antimicrobial effect after at least 25 washing cycles, to counter low challenges of microbes that might be expected to be encountered by the user of the fabric.

Hernia repair is most often preformed in an open surgical procedure. Ventral hernia repair almost always involves large abdominal openings that subject the patient to potentially large one time challenges of pathogens. Once the abdominal cavity is closed following the repair, the potentially large pathogen challenge is localized in the mesh area. Systemic antibiotic treatment is often not effective in treating the ensuing infection.

What is needed then is an antimicrobial coating for surgical mesh, and a method of manufacturing, that provides adequate localized protection against pathogens that may cause infections. What is further needed is such a non-toxic coated mesh.

### SUMMARY OF THE INVENTION

The present invention is directed to a method of preparing a surgical mesh for implantation into a body, including providing a surgical mesh to be used in the body, attaching a therapeutic amount of an antimicrobial substance to the surgical mesh, and stabilizing the surgical mesh having antimicrobial substance attached thereto.

In some embodiments, the method also includes sterilizing the stabilized surgical mesh having antimicrobial substance attached thereto.

In other embodiments, the stabilizing step further includes freeze drying the surgical mesh having antimicrobial substance attached thereto, the surgical mesh having an antimicrobial effectiveness that is the same as before being stabilized.

In yet other embodiments, the stabilizing step includes maintaining the surgical mesh at a temperature below about 4 degrees Celsius.

In another aspect, the invention is directed to a method of providing a surgical mesh having a therapeutic amount of an antimicrobial substance attached thereto at a first location, the surgical mesh originating at a second location, the second location being geographically distinct from the first location, the method includes providing a surgical mesh to be used in the body, attaching a therapeutic amount of an antimicrobial substance to the surgical mesh at the second location, stabilizing the surgical mesh having antimicrobial substance attached thereto at the second location, and preparing the stabilized surgical mesh to be shipped to the first location.

In yet another aspect, the invention is directed to a surgical mesh for implantation into a body that includes a surgical mesh to be used in the body, and a therapeutic amount of an antimicrobial substance attached to the surgical mesh, wherein the surgical mesh had the antimicrobial substance attached at least 30 days prior to the use of the surgical mesh.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description of the present embodiments of the invention, and are intended to provide an overview or framework for understanding the nature and character of the invention as it is claimed. The accompanying drawings are included to provide a further understanding of the invention, and are incorporated into and constitute a part of this specification. The drawings illustrate various embodiments of the invention and, together with the description, serve to explain the principles and operations of the invention.

### BRIEF DESCRIPTION OF THE DRAWINGS

FIG. 1 has two photographs of a microscopic view of Bard mesh prior to incubation at 40× and 100× magnifications;

FIG. 2 has two photographs of the same lysostaphin coated mesh after a 12 hour freezing cycle followed by a 12 hour lyophilization cycle;

FIG. 3. shows photographs of a microscopic view of the same mesh after the 1 hour freeze-12 hour lyophilization cycle;

FIG. 4 is a graph illustrating turbidimetric measurements of the 1 hour freeze-12 hour lyophilized mesh in 10E8 CFU/ml Staphylococcus aureus solution before and after lyophilization using the 1 hour-12 hour cycle; and

FIG. 5 is a graph illustrating turbidimetric activity of the Bard mesh prior to sterilization and after 10 and 25 kGy exposures.

### DETAILED DESCRIPTION OF THE PREFERRED EMBODIMENT

Soft tissue repair mesh in current use comprises both synthetic and biological scaffolding. Infection and foreign body response are issues that affect performance of these implant materials. The current invention discloses the attachment of various antimicrobial agents to both synthetic and biological meshes in such a way as to provide effective antimicrobial action that is often needed owing to large localized bacterial challenges resulting from large abdominal openings necessary for hernia repair, particularly ventral hernias. The antimicrobial agent must be bound to the mesh in an amount and in such a way as to allow the mesh to be inserted into the surgical field such that antibacterial action can take place both from leached (free) and bound antimicrobial molecules. The “microscopic” area of the mesh must be adequate to allow surface adsorption of ample antimicrobial agent molecules to counter the magnitude of the bacterial challenge, either through leaching, bound state action, and/or both.

There are many known antimicrobial agents in the art that might be useful as coatings for implant mesh. Among them are lysostaphin, triclosan, ethanol, LL-37 peptide, various human defensins, and combinations of these and other antibiotics. The ideal requirements are that the agents should be non-toxic and be readily attached to the mesh in such quantities as to be effective against large bacterial challenges, 10E8 CFUs/ml for example.

### Protocols for Preparation of the Preferred Embodiments

Protocol for Coating Meshes with Lysostaphin (Adsorption Protocol)

1). The mesh material was cut into 3×3 cm pieces in a laminar flow hood under sterile conditions prior to physical adsorption.

2). The samples pieces were then placed in a sterile 50 ml conical tube and incubated in 30 ml PBS buffer (10 mM phosphate; 140 mM NaCl, 3 mM KCl; pH-7.4) at room temperature (RT) for 30 minutes. As per manufacturer's instructions (Calbiochem—Cat #524650) dissolving one PBS tablet in 1 liter of deionized H2O yields 140 mM NaCl, 10 mM phosphate buffer, and 3 mM KCl, pH 7.4.

3). The solution was discarded and the samples were then gently flushed several times with PBS buffer.

4). The samples were incubated in 30 ml of PBS buffer at RT for 30 minutes and step 3 was repeated.

5). The lysostaphin (Sigma Aldrich—L7386; lyophilized powder—5 mg, Protein˜50-70%; remaining NaCl) was re-suspended in 1 ml of sterile PBS (autoclaved at 121° C.).

6). Initial lysostaphin concentrations 0.025, 0.05, 0.1, 0.25, and 0.5 mg/ml PBS buffer were prepared from a 1 mg/ml stock solution of Lysostaphin.

7). One ml of protein samples was then added to sterile (autoclaved at 121° C.) 25 ml glass vials (VWR—Cat #66012-044) in a laminar flow hood under sterile conditions.

8). The mesh samples were gently placed into each of the vials containing the protein solutions and incubated overnight at room temperature (preferably in an incubator/shaker).

9). The sample solution was removed after overnight incubation and then gently flushed several times with PBS buffer using a 1 ml pipette.

10). The samples were then stored prior to use at 4° C. in 1 ml of PBS buffer.

Protocol for Fluorescence Measurements of Lysostaphin Residual on the Mesh

Alexa Fluor 594-labeled lysostaphin is prepared according to the manufacturer's instruction. The initial fluorescence intensity of the enzyme sample solution is measured using a microplate reader (Ex 594 nm; Em 625 nm).

The mesh is then incubated in the labeled solution at room temperature for one hour with varying lysostaphin concentrations making sure the mesh is entirely covered with solution. After incubation the mesh is washed 2 times with copious amounts of PBS buffer. The samples were then added to sterile glass vials containing the enzyme solution and incubated overnight at room temperature with gentle shaking (100 rpm). The enzyme solution over the mesh was then collected and stored for fluorescence measurements, and the mesh was gently washed 2 times with 1 ml of PBS buffer. The wash solution was also collected and used in determination of enzyme residual. To remove any loosely adsorbed enzyme, 1 ml of 0.1 (v/v %) Tween 20 solution (non-ionic surfactant) is then added to the glass vials followed by incubation for 3 hours. This surfactant solution is also collected and used in the determination of the amount of desorbed enzyme, and the mesh samples are again washed with copious amount of PBS buffer. The concentration of unbound enzyme in each of the supernatants and wash solutions is determined from fluorescence measurements. The initial enzyme solution with known concentrations is used as the standards. The concentration of the unbound enzyme at each step is then calculated and subtracted from initial concentration of enzyme present in the initial solution. The difference in the concentrations corresponded to the residual enzyme concentration on the mesh. The fluorescence measurements showed zero residual on the mesh within the sensitivity of the measurements, 150 micrograms per gram of mesh. The weight of lysostaphin residual on the mesh versus initial concentration of the incubation solution is the recorded.

Table I depicts the results of fluorescence measurements of lysostaphin residual on various meshes, both biologic and synthetic, using the above protocols for incubation and measurements of lysostaphin residual on the meshes.

| | | |
|---|---|---|
| | Concentration, | Adsorption Amount, |
| Mesh (Manufacturer) | μgm/ml | μgm (μgm/cm\hat{ }2) |

Biologic Meshes (3 × 3 cm patches)

| | | | |
|---|---|---|---|
| FlexHD ® (MTF) | 25 | 56 | (6.2) |
| | 50 | 90 | (10) |
| | 100 | 224 | (25) |
| | 300 | 569 | (63) |
| | 500 | 955 | (106) |
| Strattice ® (Life Cell) | 25 | 53 | (5.9) |
| | 50 | 72 | (8.0) |
| | 100 | 146 | (16) |
| | 300 | 435 | (48) |
| | 500 | 677 | (75) |
| Permacol ® (Covidien) | 25 | 28.0 | (3.1) |
| | 50 | 65.3 | (7.3) |
| | 100 | 150 | (16.6) |
| | 300 | | |
| | 500 | 533 | (59) |
| Alloderm ®(Life Cell) | 25 | | |
| | 50 | 74.4 | (8.3) |
| | 100 | 139 | (15.4) |
| | 300 | | |
| | 500 | | |

Synthetic Meshes (3 × 3 cm patches)

| | | | |
|---|---|---|---|
| Bard ™ (CR Bard) | 25 | 42 | (4.6) |
| | 50 | 50 | (5.6) |
| | 100 | 139 | (15.4) |
| | 300 | 262 | (29) |
| | 500 | 246 | (27) |
| Parietex ® (Covidien) | 25 | 35 | (3.9) |
| | 50 | 57 | (6.3) |
| | 100 | 116 | (13) |
| | 300 | 357 | (40) |
| | 500 | 452 | (50) |

In Vivo Studies Protocol

All animal experiments were approved by the Institutional Animal Care and Use Committee (CMC) and performed in accordance with NIH guidelines. Surgical anesthesia was induced and maintained with inhaled isofluorane. The abdominal wall was shaved, prepared with Betadine® and isopropyl alcohol 70% (v/v), and draped in sterile fashion. A 1 cm midline vertical incision was made through dermis, and a pocket was then created by elevating the skin and subcutaneous tissue from the anterior abdominal fascia bilaterally. Sterile mesh 3 cm×3 cm was placed on the fascia and secured with 4-0 Prolene suture. One-by-one cm meshes were used for ex-vivo testing of anti-microbial activity. Three-by-three cm meshes were utilized for long-term studies including animal survival and gross and microscopic evaluation. Bacterial inoculation was performed by applying a 1 cc suspension of Staph aureus directly on the mesh. The incision was closed using vicryl suture and reinforced with skin staples. Topical Bitter Orange (ARC Laboratories, Atlanta, Ga.) was applied over the closure to dissuade animal-induced wound disruption. All animals received bupranorphine (0.03 mg/kg) immediately after surgery and every 12 hours thereafter for the next 48 hours.

Mesh harvest was performed surgically in a sterile fashion. General anesthesia was induced with isofluorane and the animals were euthanized by intracardiac pentobarbital injection after collecting blood samples for analysis. The skin around the original incision was prepared in the same fashion as above and opened sharply and widely to allow full exposure of the implant. The entire abdominal wall including the mesh was excised en bloc for analysis.

Table II depicts bacterial count from explanted meshes after 7 days in vivo. Four animals in each of two study arms were inculcated with 5×10E5 CFUs of Staphylococcus aureus at the time of implant. The bacteria were placed on the center of the abdominal patch prior to wound closure. Controls (No LYS) were implanted with uncoated biological and synthetic meshes. The animals in the second arm (LYS) were implanted with biological and synthetic meshes each with incubation concentrations adjusted to yield a lysostaphin concentration of 15.4 μgms/cm̂2 of mesh geometrical surface area by extrapolating the data in Table I. (For example Alloderm was incubated in 100 μgms/ml solution). A third arm of four animals with each mesh comprised implants with no lysostaphin and no bacteria (No LYS, No bacteria).

TABLE II
Average Bacterial Count after 7 Days

| | | | |
|---|---|---|---|
| | Bacterial | | Bacteria Count |
| | Count | Bacterial Count | (No LYS, |
| Mesh (Manufacturer) | (No LYS) | (LYS) | No bacteria) |

Biologic Meshes (3 × 3 cm patches)

| | | | |
|---|---|---|---|
| FlexHD ® (MTF) | 8.8 × 10E6 | 0 | 0 |
| Strattice ® (Life Cell) | 6.9 × 10E6 | 0 | 0 |
| Permacol ® (Covidien) | 1.9 × 10E8 | 0 | 0 |
| Alloderm ®(Life Cell) | 5.2 × 10E6 | 0 | 0 |

Synthetic Meshes (3 × 3 cm patches)

| | | | |
|---|---|---|---|
| Parietex ® (Covidien) | 1.5 × 10E8 | 0 | 0 |
| Bard ™ (CR Bard) | 2.8 × 10E6 | 0 | 0 |

Histology Measurements Protocol

Tissue histology was evaluated after implantation of lysostaphin-bound mesh in the presence of a large S. aureus innoculum. Mesh explantation was performed at 60 days. Specimens were prepared for light microscopy analysis. Hematoxylin and Eosin staining and Milligan's Trichrome staining were performed. The entire mesh-tissue interface on the slide was examined at low-power magnification (20×). A quantitative assessment by two blinded evaluators was made by counting the number of neutrophils and macrophages and amount of fibroblast in a fixed number of high-powered fields (40×). Histological assessment results were averaged prior to analysis (n=10).

Table III depicts the average histology results for 10 samples prepared as described above.

| Mesh | Lymphocytes | Neutrophils | Fibroblast |
|---|---|---|---|
| Strattice ® (Life Cell) | 2.0 | 1.1 | 4.25 |
| No Lys, No SA | | | |
| Strattice ® (Life Cell) | 2.0 | 1.4 | 2.1 |
| Lys, 10E6 SA | | | |
| Strattice ® (Life Cell) | 2.1 | 1.1 | 3.2 |
| Lys, 10E8 SA | | | |

The number of lymphocytes, neutrophils and amount of fibroblast for LYS coated mesh for both 10E6 and 10E8 inoculums were not significantly different from the controls (no LYS and no innoculum), p>0.05.

Protocol for Shear Strength Measurements

Sterile 3×3 cm mesh patches were implanted in the overlay method in the abdomen of 250-450 gram Sprague Dawley rats and harvested after 60 days. The study consisted of four arms: non lysostaphin coated mesh with no bacteria inoculum; meshes coated with 15.4 μgms/cm̂2 of lysostaphin with no inoculum: and two arms of lysostaphin coated meshes (15.4 μgms/cm̂2), one with 10E6 and one with 10E8 CFUs of Staphylococcus aureus respectively, n=10 for each leg. At harvest the patches were well attached to the abdominal tissue owing to tissue ingrowth. The patches were explanted along with the ingrown tissue attached underneath the patches. Individual samples were attached to a Mecmesin tensile testing device, model Multi-Test 1-i (1 kN). The underling tissue was clamped to one leg of the tester and the mesh to the other leg. The tester was set at 5 mm/min speed for removing the mesh from the tissue. The peak load (dominantly shear force) and the total energy required to separate the two components were recorded. Comparisons of the measurements were assumed to be a measure of the tissue ingrowth efficacy of the meshes with and without lysostaphin coatings and with and without bacteria inoculum.

Table IV depicts the peak load and pull-off energy (average n=10) for a biological meshes 60 days after implant.

| | | | Peak-Force, | Energy, |
|---|---|---|---|---|
| Mesh | Coating | Inoculum | N | mJ |
| Strattice ® (Life Cell) | None | None | 7.7 | 49.0 |
| Strattice ® (Life Cell) | Lysostaphin | None | 15.7 | 118 |
| Strattice ® (Life Cell) | Lysostaphin | 10E6 | 11.7 | 90.3 |
| Strattice ® (Life Cell) | Lysostaphin | 10E8 | 17.3 | 130 |

The peak load and the removal energy, and presumably the degree of in-growth, were significantly higher for the lysostaphin coated mesh samples (p<<0.05). The peak load and energy of the lysostaphin no bacteria, coated mesh samples and each set of bacteria inoculated samples were not significantly different (p>0.05). It should be noted that the above mesh is not cross-linked.

Leaching Measurement Protocol

Leaching of bound lysostaphin is an important parameter to be examined while determining the effectiveness of such antibacterial meshes. In this regard in vitro leaching was monitored over 72 hours. Leaching measurements were performed with lysostaphin coated meshes submersed in 2% (20 mg/ml) BSA-150 mM PBS solution. Meshes, placed in 25 ml amber jars, were incubated in fluorescently labeled lysostaphin solution for one hour and washed according to the adsorption protocol. Two-tenths of a ml of initial lysostaphin solution utilized for these various meshes was retained so as to obtain a standard curve to determine the leached fraction. Also, a control mesh, without lysostaphin, was incubated in 150 mM PBS solution under the same protocol as the adsorption protocol. The jars were then wrapped in aluminum foils, after adsorption, to prevent photo bleaching of fluorescently labeled enzyme. This was followed by incubation of the meshes, under mild shaking, in 3 ml of 2% BSA at 37° C. for 24-72 hrs. 0.2 ml aliquots, containing the leached enzyme, were collected at specified time points. Simultaneously, the same volume of 2% BSA-150 mM PBS was added to the meshes at each of these time points so as to maintain the existing 3 ml volume present before withdrawal of 0.1 ml from these aliquots. A standard curve was obtained for 0.1 ml of labeled enzyme; after performing a two-fold serial dilution of 1 mg/ml labeled lysostaphin using a microplate reader. (Ex 594 nm and Ex 625 nm resp). The amount of leached enzyme was obtained from the standard curve after subtracting the fluorescence value of the control from that of the sample fluorescence value at each time point. All fluorescence measurements of the standards and unknown were done simultaneously. The fraction of leached enzyme was calculated using the known amount of enzyme adsorbed onto the mesh after adsorption and plotted as a function of time.

Table V depicts the leach rate of various meshes as measured according to the above protocol.

| | | | |
|---|---|---|---|
| Mesh, (Manufacturer) (Initial | Time | Amount Leached | Percent |
| Amount Adsorbed, μgm) | Hours | (μgm) | Leached |

Biologic Meshes

| | | | |
|---|---|---|---|
| FlexHD ® (MTF) (223.7) | 0.5 | 1.4 | 0.6 |
| | 6 | 8.8 | 3.9 |
| | 12 | 15.9 | 7.1 |
| | 24 | 20.8 | |
| Strattice ® (Life Cell) (146.3) | 0.5 | 16 | 10.9 |
| | 6 | 16.3 | 11.1 |
| | 12 | 28.9 | 19.8 |
| | 24 | 23.7 | |
| Permacol ® (Covidien) (133.7) | 0.5 | 14.3 | 10.7 |
| | 5 | 18.3 | 13.7 |
| | 24 | 16.9 | 12.6 |
| | 72 | 24.3 | 18.2 |

Synthetic Meshes

| | | | |
|---|---|---|---|
| Parietex ® (Covidien) (133.7) | 0.5 | 11.2 | 6.5 |
| | 6 | 17.2 | 10 |
| | 12 | 31.0 | 18 |
| | 24 | 27.0 | 15.7 |
| Bard ™ (CR Bard) (104.7) | 0.5 | 2.1 | 2.0 |
| | 6 | 9.1 | 8.7 |
| | 12 | 19.8 | 18.9 |
| | 24 | 23.3 | 22.3 |
| Ultra Pro ® Ethicon (128.6) | 0.5 | 0.96 | 0.74 |
| | 6 | 14.2 | 11.0 |
| | 12 | 19.8 | 15.4 |
| | 24 | 18.7 | 14.5 |

Turbidimetric Activity Assay

As per the instructions of the vendor, ATCC, the Staphylococcus aureus cell suspension was grown by inoculating 15 ml of 3% (w/v) Tryptic soy with 100 μL of S. aureus culture. Mid-log phase growth was achieved by incubating the cells at 370° C. for 18-24 hours. The cells were then centrifuged at 8,000 RPM for 10 min and re-suspended in 150 mM PBS to obtain a final optical density of ˜0.6 at 600 nm. 1 mL of cells containing ˜10E8 CFU (colony forming units) was added to vials containing 1×1 mesh samples. The samples were incubated with the cell suspension at 37° C. under continuous shaking, and the rate of bacterial lysis was monitored for 24 hours by taking 0.2 mL aliquots from the reaction mixture and measuring the optical density at 600 nm in a 96 well plate at different time intervals.

Lyophilization (Freeze Drying) Protocol

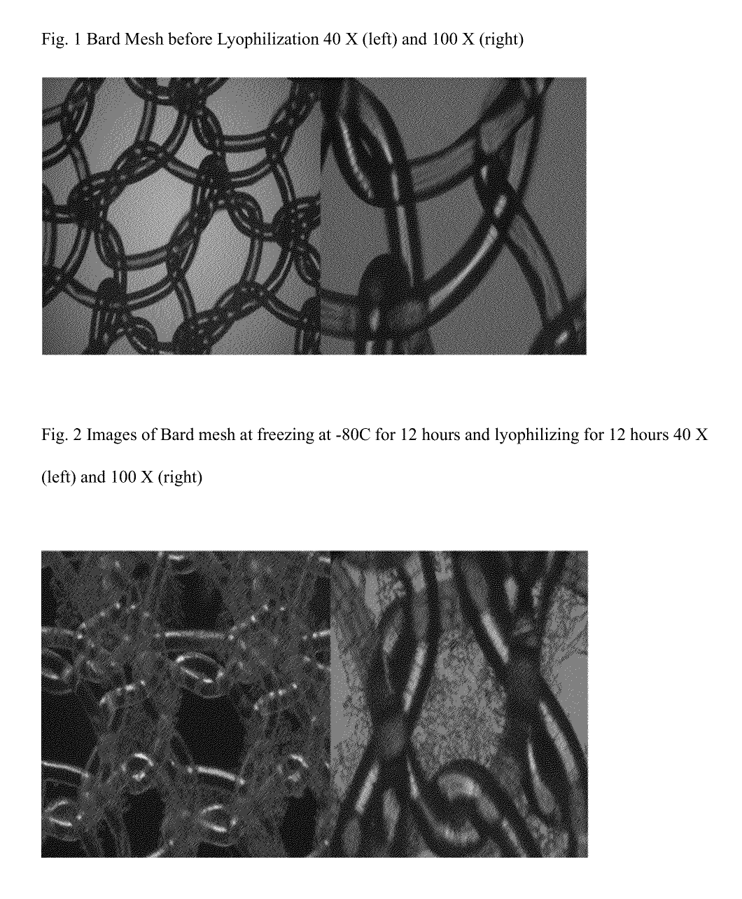

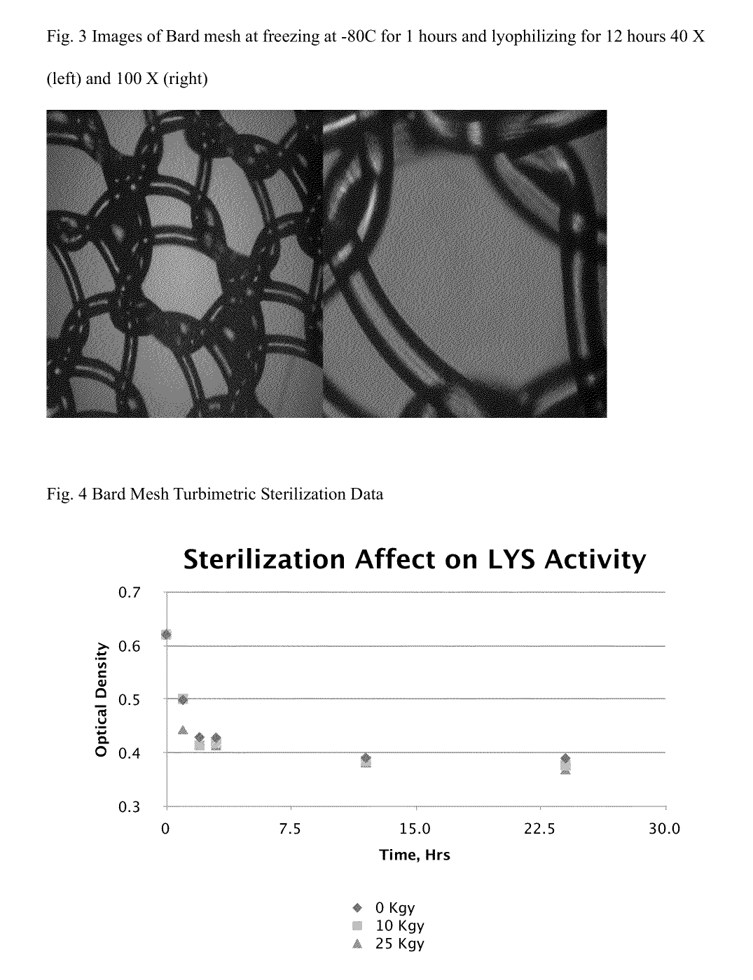

Employing a Labconco Freezone 4.5 catalog #7750000 freeze dryer, two different lysostaphin coated mesh samples were subjected to −80° C. for two different times (one sample for one hour and the other for 12 hours) before being lyophilized for 12 hours. Following this process the samples were tested for activity according to the Turbidimetric Activity Assay procedure. FIG. 1 is microscopic photographs of Bard mesh prior to incubation at 40× and 100× magnification. FIG. 2 has two photographs of the same lysostaphin coated mesh after a 12-hour freezing cycle followed by a 12-hour lyophilization cycle. The crystalline-like structures are seen surrounding the mesh filaments (polypropylene) which is believed to be residual salts from the PBS solution used in the coating process. Samples freeze dried using the 12-hour freeze and then the 12-hour lyophilization cycle showed little or no activity against Staphylococcus aureus. FIG. 3 shows pictures of the same microscopic views of the mesh after the 1-hour freeze and then the 12-hour lyophilization cycle. There is no evidence of crystallization. FIG. 4 depicts turbidimetric measurements of the 1 hour freeze-12 hour lyophilized mesh in 10E8 CFU/ml Staphylococcus aureus solution along with controls (no mesh samples) before and after lyophilization using the 1-hour-12-hour cycle.

Lysostaphin is stable for more than six (6) months when kept at 4° C. and longer at lower temperatures. Thus, an alternate to freeze drying a lysostaphin coated mesh is storing it at or below 4° C.

Sterilization Testing Protocol

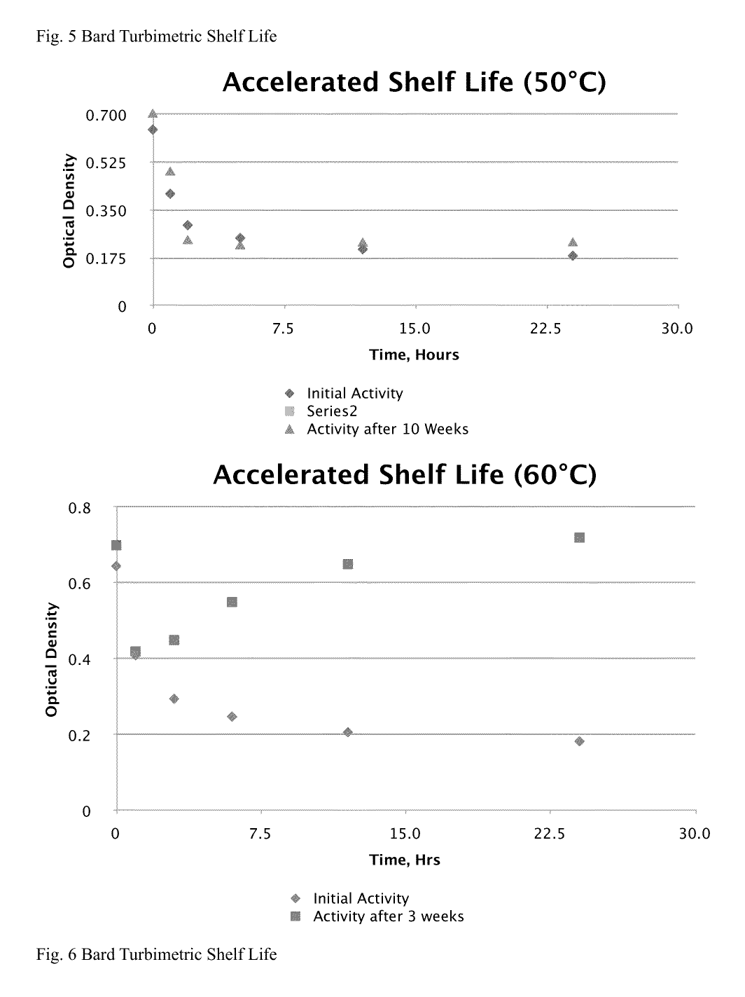

Following lyophilization using the 1 hour-12 hour cycle, mesh samples were placed into Dispos-a-vent, medical grade pouches provided by Oliver Tolas Healthcare Packaging (Hamilton, Ohio). Prior to packaging these meshes, the pouches were purged with 99% pure compressed nitrogen gas for 5 minutes and then the meshes were placed within the pouches using forceps, which were cleansed with ethanol, and the pouches were heat sealed. These pouches were sterilized using e-beam sterilization at exposure levels of 10 and 25 kGy. FIG. 5 shows turbidimetric activity of the Bard mesh prior to sterilization and after the 10 and 25 kGy exposures.

Accelerated Shelf Life Testing Protocol

Samples of lysostaphin coated Bard mesh were sealed in pouches as described above and placed into a temperature controlled oven at 50° C. for 10 weeks. The (average n=4) activity of the coated mesh samples were measured by turbidimetric activity assay as described above at various time points before and after the 10 week oven exposure (See FIG. 6). Those skilled in the art will recognize that for first order systems, 50° C. for 10 weeks exposure is equivalent to approximately 80 weeks at 20° C., depending upon the activation energy of the reaction.

Samples were also placed into a temperature controlled oven at 60° C. for 3 weeks. However, as illustrated in FIG. 6, the activity of the lysostaphin was severely affected by the temperature. It appears that the temperature was too high, disrupting the lysostaphin rather than aging the packaging in any meaningful way.

The inventors have discovered that 15.4 μgm of lysostaphin adsorbed on a square centimeter of geometrical area of implanted mesh is adequate for eradicating an innoculum of 10E8 CFUs of staphylococcus aureus in vivo using a rat model, for both synthetic and biological mesh. This surface concentration is adequate to supply wound infection protection via leaching and protection against implant infection.

The following examples are indicative of the preferred embodiments of the method of utilizing and applying this invention:

### EXAMPLE 1

Mesh samples, 3×3 cm, were coated with 15.4 μgm/cm̂2 of lysostaphin, a value arrived at by interpolating the adsorption data from Table I. The results are shown in Table VI below.

| | LYS concentration for adsorption of |
|---|---|
| Mesh (Manufacturer) | 15 μgm/cm\hat{ }2, μgm/ml |
| FlexHD ® (MTF) | 68 |
| Strattice ® (Life Cell) | 98 |
| Permacol ® (Covidien) | 86 |
| Alloderm ® (Life Cell) | 100 |
| Parietex ® (Covidien) | 118 |
| Bard ™ (CR Bard) | 100 |

Incubating the above meshes as per the coating protocol (n=10 in each study arm), using the concentrations in Table VI, and verifying the adsorbed amount by the florescence protocol above, implanting them in a rat models for 60 days with Staph A inoculums of either 10E6 or 10E8 CFUs resulted in no wound infections, no mesh infections, no residual bacteria count, and no visual or clinical effects on the rats. Of the control rats with no lysostaphin and with 10E8 inoculum 100% died or required euthanization because of wound failures prior to the 60 day study length. Thus, the lysostaphin mesh coating and the leached lysostaphin as per Table V protected the animals against mesh and wound infections.

### EXAMPLE 2

Three sets of samples of Strattice mesh (3×3 cm) were coated with 15.4 μgm/cm̂2 of lysostaphin as per the above protocol (n=10 in each study arm), and implanted in rat models. The control arm consisted of mesh without lysostaphin coating and the two coated arms were inoculated with 10E6 and 10E8 CFUs of Staph A. All samples were harvested after 60 days. Both lysostaphin arms showed significantly higher pull-off strength from the underlying tissue compared to those in the control arm, thus indicating that the coated meshes encouraged better tissue ingrowth when tested as per the above protocol. In addition, histology cell counts as per the above protocol showed no significant differences between the three arms.

### EXAMPLE 3

Mesh samples were freeze dried with the 1 hour freeze and 12 hour drying cycle as described above following coating with lysostaphin. The Turbidimetric Activity Assay before and after the lyophilization were not significantly different indicating that this freeze/dry cycle preserves lysostaphin coated mesh activity against Staph A.

### EXAMPLE 4

Lysostaphin coated mesh samples were lyophilized as per the protocol above, packaged as described above and radiation sterilized at 10 and 25 kGy. Turbidimetric Activity Assay was made before and after sterilization with no significant difference between the pre and post sterilization samples indicating that radiation sterilization of this magnitude has no effect on the coated lysostaphin mesh activity against Staph A.

### EXAMPLE 5

Lysostaphin coated mesh samples were freeze dried, packaged, and sterilized as per the above protocols and placed into a 50° C. oven for 10 weeks. Turbidimetric Activity Assay of pre and post oven samples was performed and no significant difference in activity was found. This indicates that the shelf life of sealed, sterilized lysostaphin coated meshes have a shelf life greater than 1 year.

### EXAMPLE 6

Immediately following the buffer solution coating process, the surgical mesh was packaged as above, and was kept at 4° C. or lower and sterilized by electron beam. The packaged mesh was then stored and a Turbidimetric Activity Assay of pre and post storage of these samples was performed and no significant difference in activity was found in the pre and the post stored samples. It should be noted that while electron beam sterilization was used in this example, other sterilization radiation (e.g., gamma), could be used as well.

Typically, the effectiveness of the antimicrobial properties only last for a short period of time (usually measured in hours), meaning that the surgical mesh would have to be prepared either on-site (even in the operating room) or very close by for the coated surgical mesh to be effective. But since it has been discovered that the effectiveness can be preserved with both of the stabilization methods noted above, either freeze-dried or maintained at least at about 4° C., the coated surgical mesh can be prepared off-site and prior to it being needed and then shipped to a destination for use in surgery after the stabilization of the mesh.

And it will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

We claim:

1. A method of preparing a surgical mesh for implantation into a body comprising:
providing a surgical mesh to be used in the body;
attaching a therapeutic amount of an antimicrobial substance to the surgical mesh; and
stabilizing the surgical mesh having antimicrobial substance attached thereto.

2. The method of preparing a surgical mesh according to claim 1, further comprising:
sterilizing the stabilized surgical mesh having antimicrobial substance attached thereto.

3. The method preparing a surgical mesh according to claim 1, wherein the stabilizing step further comprises freeze drying the surgical mesh having antimicrobial substance attached thereto, the surgical mesh having an antimicrobial effectiveness that is the same as before being stabilized.

4. The method preparing a surgical mesh according to claim 1, wherein the freeze drying includes the steps of:
freezing the surgical mesh having antimicrobial substance attached thereto for a first predetermined time;
drying the surgical mesh having antimicrobial substance attached thereto for a second predetermined time.

5. The method preparing a surgical mesh according to claim 4, wherein the first predetermined time is less than 12 hours and the second predetermined time is at least 12 hours.

6. The method preparing a surgical mesh according to claim 4, wherein the first determined time is about an hour and the second predetermined time is about 12 hours.

7. The method of preparing a surgical mesh according to claim 2, wherein the step of sterilizing the stabilized surgical mesh includes exposure to one of gamma radiation and an electron beam.

8. The method of preparing a surgical mesh according to claim 1, wherein the step of stabilizing comprises maintaining the surgical mesh at a temperature below about 4 degrees Celsius.

9. A surgical mesh prepared according to the method of claim 1, the surgical mesh having a shelf-life of at least six months.

10. A method of providing a surgical mesh having a therapeutic amount of an antimicrobial substance attached thereto at a first location, the surgical mesh originating at a second location, the second location being geographically distinct from the first location, the method comprising:
providing a surgical mesh to be used in the body;
attaching a therapeutic amount of an antimicrobial substance to the surgical mesh at the second location;
stabilizing the surgical mesh having antimicrobial substance attached thereto at the second location; and
preparing the stabilized surgical mesh to be shipped to the first location.

11. The method according to claim 10, wherein the step of preparing includes sterilizing the stabilized surgical mesh having antimicrobial substance attached thereto.

12. The method according to claim 10, wherein the stabilizing step further comprises freeze drying the surgical mesh having antimicrobial substance attached thereto, the surgical mesh having an antimicrobial effectiveness that is the same as before being stabilized.

13. The method preparing a surgical mesh according to claim 12, wherein the freeze drying includes the steps of:
freezing the surgical mesh having antimicrobial substance attached thereto for a first predetermined time;
drying the surgical mesh having antimicrobial substance attached thereto for a second predetermined time.

14. The method preparing a surgical mesh according to claim 13, wherein the first predetermined time is less than 12 hours and the second predetermined time is at least 12 hours.

15. The method preparing a surgical mesh according to claim 13, wherein the first determined time is about an hour and the second predetermined time is about 12 hours.

16. The method of preparing a surgical mesh according to claim 11, wherein the step of sterilizing the stabilized surgical mesh includes exposure to one of gamma radiation and an electron beam.

17. A surgical mesh for implantation into a body comprising:
a surgical mesh to be used in the body; and
a therapeutic amount of an antimicrobial substance attached to the surgical mesh,
wherein the surgical mesh had the antimicrobial substance attached at least 30 days prior to the use of the surgical mesh.

18. The surgical mesh according to claim 17, wherein the surgical mesh has been freeze-dried.

19. The surgical mesh according to claim 17, wherein the surgical mesh has been maintained at a temperature of about 4 degrees Celsius.

20. The surgical mesh according to claim 17, wherein the surgical mesh has been prepared in a place other than where it will be implanted.
